(19) United States
(12) Patent Application Publication
(10) Pub. No.: US 2024/0238492 A1
(43) Pub. Date: Jul. 18, 2024

(54) AN APPARATUS FOR EXTRACORPOREAL BLOOD TREATMENT

(71) Applicant: GAMBRO LUNDIA AB, Lund (SE)

(72) Inventors: Paolo ROVATTI, Finale Emilia (IT); Alessandro SURACE, Carpi, Modena (IT)

(21) Appl. No.: 18/565,022

(22) PCT Filed: May 10, 2022
(86) PCT No.: PCT/EP2022/062574

§ 371 Date: Nov. 28, 2023

(30) Foreign Application Priority Data
May 31, 2021 (EP) 21176784.3

(51) Int. Cl. A61M 1/16; A61M 1/34; G16H 40/63

(52) U.S. Cl. CPC A61M 1/1609; A61M 1/1607; A61M 1/1611; A61M 1/3403; A61M 1/3413; G16H 40/63; A61M 2205/3317; A61M 2230/20

(57) ABSTRACT

A control unit of an apparatus for extracorporeal blood treatment is configured to perform a control procedure. The control procedure includes calculating parameter control values to be set during a time interval after the instant in which the control is made, on the basis of actual values and of prescription values of variation in blood volume, ultrafiltration flow rate, and concentration of sodium. The control procedure further includes imposing the parameter control values during the time interval consecutive to the instant in which the control is made such that the actual values of the variation in blood volume track the prescription values of the variation in blood volume over a predetermined treatment time. The control unit is further configured to perform a procedure for automatically estimating/calculating a target plasma conductivity and/or a target concentration of sodium in plasma to be reached at the end of the extracorporeal blood treatment.

The present invention relates to an apparatus for extracorporeal blood treatment.

An apparatus for extracorporeal blood treatment comprises at least one treatment unit (for example a dialyser or a filter or ultrafilter or a plasma filter or a filtering unit of another type) having a semipermeable membrane which separates the treatment unit into two chambers. An extracorporeal blood circuit enables circulation of blood removed from a patient internally of the first chamber. At the same time and typically in a counter-current direction with respect to the blood, a treatment fluid is made to circulate through an appropriate circuit in the second chamber of the treatment unit. This type of apparatus for blood treatment, known as dialysis apparatus, may be used for removal of excess solutes and fluids from the blood of patients suffering from kidney failure. A particular type of apparatus for blood treatment, known as hemofiltration or hemodiafiltration apparatus, further comprises one or more infusion lines predisposed to send a replacement fluid into the extracorporeal blood circuit. The infusion line or lines are connected upstream and/or downstream with respect to the treatment unit. The above-described blood treatment apparatus may be controlled in various ways during patient treatment.

Document WO 2012/172398 A1 discloses an apparatus for extracorporeal blood treatment comprising sensors for determining a first parameter relating to a patient's blood volume, a second parameter relating to an ultrafiltration flow rate or to a patient's weight loss rate, a third parameter relating to a conductivity or concentration of a liquid crossing the dialysis line and/or the infusion line and a fourth parameter relating to an infusion flow rate. The apparatus comprises a control unit for performing a control procedure comprising: receiving, from the sensors, the measured values of the above-cited parameters and calculating, on the basis of the measured values and the prescription values of the variation in blood volume, the weight loss, the plasma conductivity or sodium concentration, the infusion volume, control values to be imposed during a time interval following the control instant.

WO 2012/172398 uses the sodium concentration in dialysis fluid to increase the extra cellular osmolality with the goal to trigger and sustain the water shift from the intracellular to the extracellular compartment (i.e. plasma refilling), just as needed.

The Applicant observed that dialysate sodium prescriptions are mostly independent of pro-dialysis serum sodium levels and this lack of individualization may lead to dialysis that ignores a sodium set point of the patient, causing unwanted deviation in terms of patient fluid balance.

The Applicant observed that the prescription values of plasma conductivity and/or sodium concentration to be achieved in the patient in the treatment time T, i.e. at the end of the extracorporeal blood treatment, is/are set by the operator but is/are not known a priori.

Indeed, for instance, WO 2012/172398 discloses only that the prescription values of plasma conductivity or sodium concentration are entered through a user interface and received by the control unit and then used to calculate desired progressions over time.

Therefore, the prescription value of sodium concentration is usually measured periodically (e.g. once per month) through an expensive laboratory test and adapted month by month on the base of the last laboratory data or may be estimated, treatment by treatment, relying on the doctor's knowledge of that specific patients hystory and pathologies.

Other existing techniques to estimate the initial patient's sodium in blood and/or to target the dialysis prescription are known but they are expensives, meaning that they require dedicated disposable (eg: hemofilters) or additional work by the operator.

Document WO 2018/095694A1 is also known comprising a control unit configured for setting a sodium concentration in the dialysis fluid and after setting the dialysis fluid at the initial set point, circulating the dialysis fluid and/or the substitution fluid, measuring an initial conductivity value of the dialysate at the beginning of the treatment, and calculating, based on the measured initial conductivity value of the spent dialysis fluid and on the corresponding conductivity value of the dialysis fluid, the value of the initial plasma conductivity. The sodium concentration set point is determined based on the sodium concentration value for an isoconductive dialysis corrected based on an adjustment term to achieve an isotonic, isonatric, or isonatrikalernic dialysis.

### SUMMARY

An aim of the present invention is to make available an apparatus for blood treatment which is able to improve the comfort of the patient during and after treatment.

An aim of the present invention is to provide an apparatus for blood treatment which is able to substantially maintain/achieve the correct plasma conductivity and/or sodium concentration in the blood of the patient at the end of the blood treatment.

An aim of the present invention is to provide an apparatus for blood treatment configured to set in quick, simple and reliable manner the prescription values of plasma conductivity and/or sodium concentration to be achieved in the patient at the end of the blood treatment.

An aim of the present invention is to provide an apparatus for blood treatment configured to reduce the workload of the operator and/or of the physician at the start of the treatment.

A further aim of the present invention is to make available an apparatus which is able to continuously modulate UF (or weight loss) and plasma conductivity and/or sodium concentration during the extracorporeal blood treatment, in order to get the best possible compromise between reaching the prescription targets and improving the comfort of the patient during treatment in a more physiological way.

A further aim of the present invention is to provide an apparatus which allows accelerating the search sequence for the wanted trajectories of prescription parameters and is however able to operate safely.

At least one of the above-indicated aims is substantially attained by an apparatus for blood treatment as in one or more of the appended claims.

Some aspects of the invention are now described.

In a 1^{st} aspect, an apparatus for extracorporeal blood treatment comprises:
a treatment unit having a first chamber and a second chamber separated from one another by a semipermeable membrane;
a blood removal line connected to an inlet port of the first chamber and predisposed to remove blood from a patient,
a blood return line connected to an outlet port of the first chamber and predisposed to return treated blood to the patient;
the blood removal line, the blood return line and the first chamber being part of an extracorporeal blood circuit;
at least a liquid evacuation line connected to an outlet port of the second chamber;
a supply line connected to an inlet port of the second chamber and/or connected to the extracorporeal blood circuit; sensor devices for detecting actual values of parameters of the extracorporeal blood treatment;
a control unit connected with the sensor devices and configured to receive prescription values of said parameters to be reached in the patient or to follow over a treatment time and to obtain, through the sensor devices, the actual values of said parameters during the extracorporeal blood treatment; wherein said parameters comprises:
   a first parameter relating to a variation in blood volume of the patient;
   a second parameter relating to an ultrafiltration flow rate through the semipermeable membrane or to a weight loss rate of the patient or to an accumulated weight loss;
   a third parameter chosen in a group consisting of: conductivity of a liquid crossing the supply line, a conductivity-related parameter of the liquid crossing the supply line, concentration of at least sodium in the liquid crossing the supply line and a concentration-related parameter of at least sodium in the liquid crossing the supply line;
wherein the control unit is configured to perform, at temporally consecutive control instants, a control procedure comprising:
   calculating, on the basis of the actual values and of the prescription values, the following control values to be set during a time interval after the instant in which the control is made:
      a second parameter control value; and
      a third parameter control value;
   imposing at least one of the second parameter control value and third parameter control value during the time interval consecutive to the instant in which the control is made such that the actual values of the first parameter track the prescription values of the first parameter over the treatment time;
wherein the prescription value of the third parameter comprises a target plasma conductivity and/or a target concentration of at least sodium in plasma to be reached in the patient in the treatment time; and
wherein the control unit is further configured to perform, at least at the beginning of the extracorporeal blood treatment, a procedure for estimating said target plasma conductivity and/or said target concentration of at least sodium in plasma.

In a 2^{nd} aspect, it is provided a procedure for estimating a target plasma conductivity and/or a target concentration of at least sodium in plasma to be reached in a patient at the end of an extracorporeal blood treatment performed by an extracorporeal blood treatment apparatus;
wherein the extracorporeal blood treatment apparatus comprises:
a treatment unit having a first chamber and a second chamber separated from one another by a semipermeable membrane;
a blood removal line connected to an inlet port of the first chamber and predisposed to remove blood from a patient,
a blood return line connected to an outlet port of the first chamber and predisposed to return treated blood to the patient;
the blood removal line, the blood return line and the first chamber being part of an extracorporeal blood circuit; at least a liquid evacuation line connected to an outlet port of the second chamber;
a supply line connected to an inlet port of the second chamber and/or connected to the extracorporeal blood circuit sensor devices for detecting actual values of parameters of the extracorporeal blood treatment;
a control unit connected with the sensor devices and configured to receive prescription values of said parameters to be reached in the patient or to follow over a treatment time and to obtain, through the sensor devices, the actual values of said parameters during the extracorporeal blood treatment; wherein said parameters comprises:
   a first parameter relating to a variation in blood volume of the patient;
   a second parameter relating to an ultrafiltration flow rate through the semipermeable membrane or to a weight loss rate of the patient or to an accumulated weight loss;
   a third parameter chosen in a group consisting of: conductivity of a liquid crossing the supply line, a conductivity-related parameter of the liquid crossing the supply line, concentration of at least sodium in the liquid crossing the supply line and a concentration-related parameter of at least sodium in the liquid crossing the supply line.

In a 3^{rd} aspect according to aspect 1 or 2, the apparatus comprises a preparation line of the liquid crossing the supply line located upstream of and connected to the supply line and the procedure for estimating comprises'
setting an initial set point of the third parameter of the liquid crossing the supply line and preparing a corresponding liquid through the preparation line;
after setting the initial set point, starting the extracorporeal blood treatment by circulating the liquid through the supply line and by circulating blood through the extracorporeal blood circuit;
measuring an initial value of the third parameter of the dialysate in the liquid evacuation line at the beginning of the extracorporeal blood treatment;
calculating, based on the measured initial value of the third parameter of the dialysate in the liquid evacuation line, an initial value of plasma conductivity and/or an initial value of concentration of at least sodium in plasma;
setting the initial value of plasma conductivity as the target plasma conductivity and/or setting the initial value of concentration of at least sodium in plasma as the target concentration of at least sodium in plasma.

In a 4^{th} aspect according to aspect 3, calculation of the initial value of plasma conductivity and/or the initial value of concentration of at least sodium in plasma is based also on the initial set point of the third parameter of the liquid crossing the supply line.

In a 5^{th} aspect according to aspect 3 or 4, the procedure for estimating further comprises:
measuring at least an initial value of the third parameter of the liquid crossing the supply line at the beginning of the extracorporeal blood treatment; said measuring being executed by receiving a signal from a sensor in the supply line;
calculating the initial value of plasma conductivity and/or the initial value of concentration of at least sodium in plasma being based also on the measured initial value of the third parameter of the liquid crossing the supply line.

In a 6^{th} aspect according to aspect 3, 4 or 5, the procedure for estimating further comprises:
applying a conductivity adjustment factor to the initial value of plasma conductivity to obtain a corrected value of plasma conductivity and/or optionally applying a concentration adjustment factor to the initial value of concentration of at least sodium in plasma to obtain a corrected value of concentration of at least sodium in plasma; and
setting the corrected value of plasma conductivity as the target plasma conductivity and/or setting the corrected value of concentration of at least sodium in plasma as the target concentration of at least sodium in plasma.

In a 7^{th} aspect according to any of aspects 3 to 6, the procedure for estimating comprises: after calculating the initial value of plasma conductivity, the control unit is configured to drive a liquid regulating device for regulating a composition of the liquid crossing the supply line to change said composition of the liquid crossing the supply line to reach a liquid conductivity substantially equal to the initial value of plasma conductivity (isoconductivity).

In an 8^{th} aspect according to any of aspects 3 to 7, the initial set point of the third parameter is set by an operator.

In a 9^{th} aspect according to any of aspects 3 to 7, the initial set point of the third parameter is calculated to match a first estimate of the plasma conductivity of the patient.

In a 10^{th} aspect according to any of aspects 3 to 7 or 9, the initial set point of the third parameter is calculated as a function of the type of extracorporeal blood treatment mode, optionally hemodiaysis (HD), hemofiltration (HF) or hemodiafiltration (HDF).

In 11^{th} aspect according to any of aspects 3 to 10, the initial set point of the third parameter is the initial set point of sodium concentration in the liquid crossing the supply line, wherein the control unit is configured to calculate the initial set point of sodium concentration in the liquid crossing the supply line as a function of the concentration of at least a further substance in the liquid crossing the supply line or as a function of an estimated plasma concentration of at least a substance or as a function of the weighted difference in concentration of at least a further substance in the liquid crossing the supply line and in the plasma or as a function of the molar conductivities of at least a substance in the liquid crossing the supply line.

In 12^{th} aspect according to any of aspects 3 to 11, the control unit is configured to calculate the initial set point of the third parameter in the liquid crossing the supply line as a function of at least one flow rate, optionally the dialysate flow rate at the outlet of the second chamber and/or as a function of at least an efficiency parameter of the treatment unit, in particular a clearance of the treatment unit, optionally an urea clearance.

In a 13^{th} aspect according to any of aspects 3 to 12, measuring the initial value of the third parameter of the dialysate comprises measuring a plurality of consecutive initial values of the third parameter until said values are stabilized to provide the initial value of the third parameter of the dialysate in stable conditions.

In a 13^{th} bis aspect according to aspects 13, the consecutive initial values of the third parameter are stabilized when they are substantially constant over time, i.e. when said values substantially do not change anymore over time.

In a 14^{th} aspect according to any of aspects 3 to 13, the control unit is configured to measure the initial value of the third parameter of the dialysate as soon as the exchange process in the treatment unit reaches stable conditions; the control unit being configured to determine reaching of stable conditions for the exchange process.

In a 15^{th} aspect according to previous aspect 14, during the step of determining reaching of stable conditions, the control unit is configured to prevent changes in a flow rate of the liquid crossing the supply line.

In a 15^{th} bis aspect according to aspect 14 or 15, the stable conditions of the exchange process means that the exchange rate/s of fluids and/or substances across the semipermeable membrane in the treatment unit is/are not subject to abrupt fluctuations. In a 16^{th} aspect according to to any of aspects 3 to 15, calculation of the initial value of plasma conductivity is performed as a function of a dialysate flow rate at the outlet of the second chamber and/or as a function of the blood flow rate in the blood circuit and/or as a function of at least an efficiency parameter of the treatment unit, optionally a clearance of the treatment unit, optionally an urea clearance.

In a 17^{th} aspect according to aspect 3 or 16, calculation of the initial value of plasma conductivity is performed according to the following formula:

$${κ}_{p , 1}^{′} = {κ}_{0 , d o} + \frac{{Q}_{d o}}{{Q}_{Bset}} ( {κ}_{0 , do} - {κ}_{0 , di} )$$

wherein

| | |
|---|---|
| κ_{p, 1} | plasma conductivity first estimate; |
| Q_{do} | dialysate flow rate at the treatment unit outlet; |
| Q_{bset} | set blood flow rate at the treatment unit inlet; |
| [symbol] _{0, di} | liquid conductivity at the treatment unit inlet |
| | for a pure electrolyte solution; |
| [symbol] _{0, do} | dialysate conductivity at the treatment unit |
| | outlet for a pure electrolyte solution. |

In a 18^{th} aspect according to aspect 3 or 17, calculation of the initial value of plasma conductivity is performed according to the following formula:

$${κ}_{p , 1}^{″} = {κ}_{0 , d i} + \frac{{Q}_{d o}}{{K}_{u}} ( {κ}_{0 , do} - {κ}_{0 , di} )$$

wherein:

| | |
|---|---|
| κ_{p, 1} | plasma conductivity first estimate; |
| Q_{do} | dialysate fluid flow rate at the treatment unit outlet; |
| Kᵤ | treatment unit clearance for urea; |
| [symbol] _{0, di} | liquid conductivity at the treatment unit inlet for a |
| | pure electrolyte solution; |
| [symbol] _{0, do} | dialysate conductivity at the treatment unit outlet for |
| | a pure electrolyte solution. |

In a 19^{th} aspect according to any of aspects 3 to 18, calculation of the initial value of plasma conductivity and/or the initial value of concentration of at least sodium in plasma is done with an iterative method.

In a 20^{th} aspect according to aspect 19, the procedure for estimating comprises: calculating a first estimate of the initial value of plasma conductivity, wherein the control unit is configured to drive a liquid regulating device for regulating a composition of the liquid crossing the supply line to change the composition of the liquid crossing the supply line to reach a liquid conductivity substantially equal to the first estimate of the initial value of plasma conductivity.

In a 21^{st} aspect according to previous aspect 20, the procedure for estimating comprises: after setting the liquid conductivity substantially equal to the first estimate of the initial value of plasma conductivity, the control unit is configured to execute a second calculating step, based on a second determined initial conductivity of the dialysate and on a second corresponding conductivity of the liquid crossing the supply line, of a second estimate of the initial value of plasma conductivity.

In a 22^{nd} aspect according to aspect 21, said calculating the second estimate is performed maintaining the liquid conductivity substantially constant and substantially equal to the calculated plasma conductivity; wherein, after calculating the second estimate of the initial value of plasma conductivity, the control unit is configured to drive the liquid regulating device to change the composition of the liquid crossing the supply line and to set the liquid conductivity substantially equal to said second estimate.

In a 23^{rd} aspect according to any of aspects 3 to 22, the initial value of concentration of at least sodium in plasma (i.e. the target concentration of at least sodium in plasma) is derived from the initial value of plasma conductivity (i.e. the target plasma conductivity).

In a 24^{th} aspect according to previous aspect 23, the initial value of concentration of at least sodium in plasma (i.e. the target concentration of at least sodium in plasma) is the initial value of concentration of sodium in plasma (or target concentration of sodium in plasma).

In a 25^{th} aspect according to the previous aspect 24, the target concentration of sodium in plasma is set as a concentration of sodium in the liquid crossing the supply line when the liquid conductivity matches the initial value of plasma conductivity.

In a 26^{th} aspect according to aspect 24, the target concentration of sodium in plasma is set as a sum of the concentration of sodium in the liquid crossing the supply line when the liquid conductivity matches the initial value of plasma conductivity (main contribution term) and of a concentration adjustment factor.

In a 27^{th} aspect according to aspect 26, the main contribution term affects the initial value of concentration of sodium in plasma for at least 80%, optionally at least 90%, of the initial value of concentration of sodium in plasma; the concentration adjustment factor contributes to the initial value of concentration of sodium in plasma for less than 15%, optionally at least 10%, of the initial value of concentration of sodium in plasma.

In a 28^{th} aspect according to aspect 26 or 27, the concentration adjustment factor is a sodium concentration adjustment relative to an isoconductive state to provide a treatment chosen in the group including isotonic dialysis, isonatric dialysis, and isonatikalemic dialysis.

In a 29^{th} aspect according to any of the previous aspects 1 to 28, the control procedure starts when the procedure for estimating is completed.

In a 30^{th} aspect according to any of aspects 1 to 29, at least one time during the extracorporeal blood treatment, the control unit is configured to check plasma conductivity and/or concentration of at least sodium in plasma.

In a 31^{st} aspect according to aspect 30, checking the plasma conductivity and/or the concentration of at least sodium in plasma comprises: measuring a value of the third parameter of the dialysate in the liquid evacuation line, optionally measuring a value of the third parameter of the liquid crossing the supply line, and calculating, based on the measured value of the third parameter of the dialysate in the liquid evacuation line and optionally on the measured value of the third parameter of the liquid crossing the supply line, a value of plasma conductivity and/or a value of concentration of at least sodium in plasma.

In a 32^{nd} aspect according to any of the previous aspect 31, checking the plasma conductivity and/or the concentration of at least sodium in plasma comprises: measuring a value of the third parameter of the liquid crossing the supply line and calculating the value of plasma conductivity and/or the value of concentration of at least sodium in plasma as a function also of the measured value of the third parameter of the liquid crossing the supply line.

In a 33^{rd} aspect according to any of aspects 1 to 32, the control procedure comprises the following sub-steps:
receiving the target plasma conductivity and/or the target concentration of at least sodium in plasma;
receiving the treatment time;
determining, on the basis of the target plasma conductivity and/or on the base of the target concentration of at least sodium in plasma, a third parameter band delimited between an upper trajectory and a lower trajectory over the treatment time or a third parameter trajectory over the treatment time.

In a 34^{th} aspect according to the previous aspect 32, the control procedure is configured to keep the calculated third parameter control values within the third parameter band or in a neighborhood of the third parameter trajectory; optionally the control procedure is configured to keep the calculated third parameter control values less than or equal to the upper trajectory of the third parameter band and greater than or equal to the lower trajectory of the third parameter band.

In a 35^{th} aspect according to any of aspects 1 to 34, the prescription values of the first parameter define a first parameter trajectory and tracking the prescription values of the first parameter comprises: keeping or moving the actual values of the first parameter on said first parameter trajectory or in a neighborough of said first parameter trajectory; wherein the prescription values of the second parameter define a second parameter band delimited between an upper trajectory and a lower trajectory over the treatment time.

In a 36^{th} aspect according to aspect 35, calculating the second parameter control value comprises:
determining at least a first error parameter on the basis of
   a difference between an actual value of the first parameter at the control instant t and a corresponding value on the first parameter trajectory; and
   a difference between an actual value of the second parameter at the control instant t and at least a corresponding value of the second parameter band;
calculating the second parameter control value on the basis of the first error parameter and of the actual value of the second parameter relating to a preceding control instant.

In a 37^{th} aspect according to any of aspects 35 or 36 in combination with aspect 33 or 34, calculating the third parameter control value comprises:
determining at least a second error parameter on the basis of:
   a difference between an actual value of the first parameter at the control instant t and a corresponding value on the first parameter trajectory; and
   a difference between an actual value of the third parameter at the control instant t and at least a corresponding value of the third parameter band or third parameter trajectory;
calculating the third parameter control value on the basis of the second error parameter and of the actual value of the third parameter relating to a preceding control instant.

In a 38^{th} aspect according to any of aspects 1 to 37, near the end of the extracorporeal blood treatment, the control procedure comprises: driving the third parameter control values to converge towards the target plasma conductivity and/or the target concentration of at least sodium in plasma.

In a 39^{th} aspect according to any of aspects 1 to 38, at the end of the extracorporeal blood treatment, the control procedure comprises: setting the third parameter control value close or substantially equal to the target plasma conductivity and/or the target concentration of at least sodium in plasma.

In a 40^{th} aspect according to any of aspects 1 to 39, the actual values of the third parameter are values of concentration of at least sodium in the liquid crossing the supply line; wherein the control procedure uses a mathematical model, representing kinetics of the solutes in a distribution volume in the patient, in order to determine equivalent sodium concentration values, wherein by equivalent sodium concentration at instant t is intended the constant sodium concentration in the liquid crossing the supply line which, if it were applied at the start of treatment up to an instant t, would lead to the same plasma sodium concentration in the patient as is obtained at the same instant t with the variation in sodium concentration or conductivity imposed by the control procedure up to time t; the control procedure using the equivalent sodium concentration values as actual values of the third parameter for the determination of the control values.

In a 41^{st} aspect according to any of aspects 1 to 40, the apparatus comprises:
a regulating device for regulating the second parameter, the regulating device being connected to the control unit and being active on at least one of the extracorporeal blood circuit and the liquid evacuation line,
a liquid regulating device for regulating a composition of the liquid crossing the supply line, the liquid regulating device being connected to the control unit and being active on the supply line for regulating the third parameter.

In a 42^{nd} aspect according to previous aspect 41, the step of imposing the control values during the procedure comprises'
commanding the regulating device to impose the second parameter control value;
commanding the liquid regulating device to impose the third parameter control value.

Further characteristics and advantages of the present invention will better emerge from the detailed description that follows of at least an embodiment of the invention, illustrated by way of non-limiting example in the accompanying figures of the drawings.

### DESCRIPTION OF THE DRAWINGS

The invention will be described with the aid of the figures of the drawings, by way of non-limiting example, which illustrate some aspects of the invention.

In particular:

FIG. 1 is a schematic illustration of an example of an extracorporeal blood treatment apparatus of the invention;

FIG. 2 is a block diagram relating to an embodiment of a control procedure of the invention;

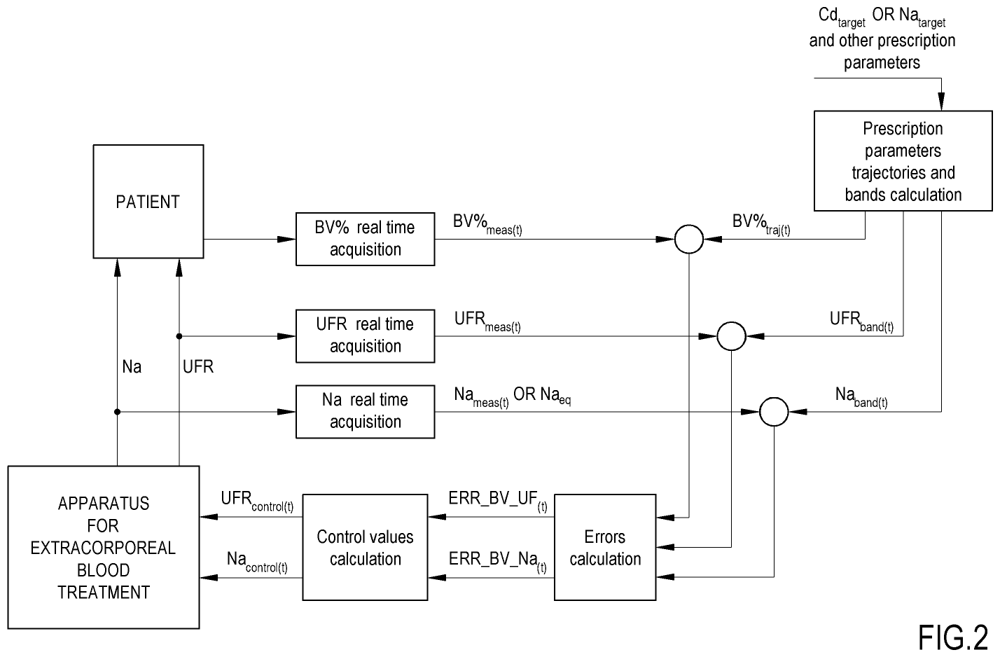

FIGS. 3A to 3C show the progression of parameters of the extracorporeal blood treatment controlled through the control procedure of the invention;

FIG. 4 is a flow diagram relating to an embodiment of an estimating procedure of the invention;

FIG. 5 is a flow diagram relating to an embodiment of a control procedure of the invention.

### DETAILED DESCRIPTION

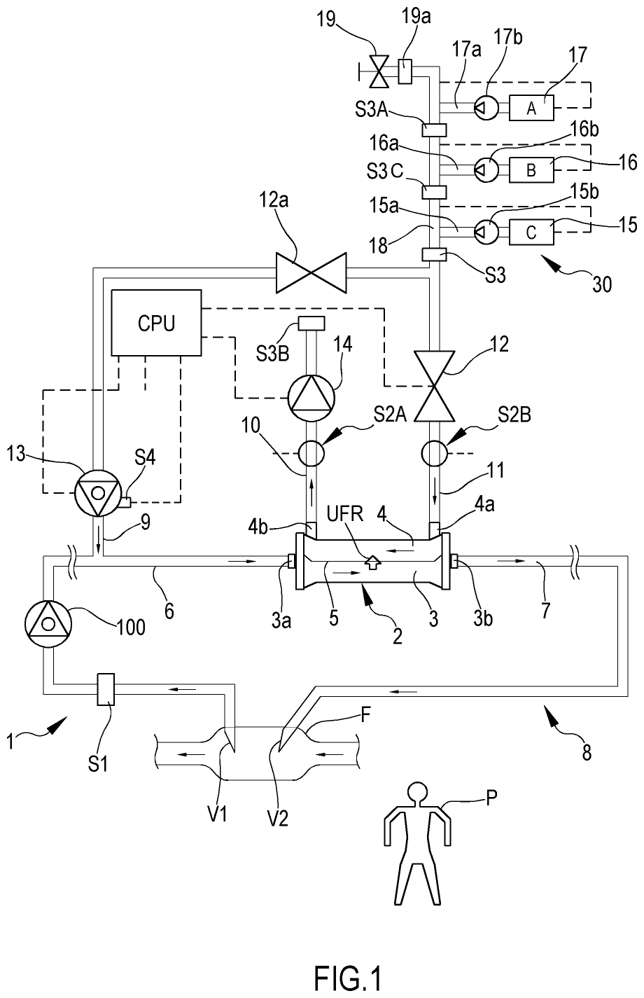

With reference to FIG. 1, number 1 denotes in its entirety an apparatus for extracorporeal blood treatment. The apparatus 1 comprises at least one treatment unit 2, for example a hemofilter, a hemodiafilter, a plasma filter, having at least one first chamber 3 and at least one second chamber 4 separated from one another by a semipermeable membrane 5.

A blood removal line 6 is connected with an inlet port 3a of the first chamber 3 and is predisposed, in operating conditions of connection to a patient P, to remove blood from a vascular access V1 inserted for example in a fistula F of the patient P.

A blood return line 7 connected to an outlet port 3b of the first chamber 3 is predisposed to receive the treated blood from the treatment unit 2 and to return the treated blood to a further vascular access V2 connected with the patient's fistula. Note that the configuration of the vascular access may be of any nature: for example a catheter, a port implanted in the patient, a cannula, a needle, etc. The blood removal line 6, the first chamber 3 of the treatment unit 2 and the blood return line 7 to the patient P are part of an extracorporeal blood circuit 8. A blood pump 100 on the blood removal line 6, during the use of the apparatus 1, provides for the circulation of the blood externally of the patient's body when subjected to treatment.

In the example of FIG. 1, an infusion line 9 of a replacement liquid is connected to the blood removal line 6, upstream of the first chamber 3. Alternatively, the infusion line 9 might be connected to the return line 7, downstream of the first chamber 3. In other examples, an infusion line may be connected downstream while another infusion line may be connected upstream of the unit 2. Further infusion lines may also be provided, for example connected downstream and/or upstream of the treatment unit 2.

The apparatus 1 further comprises at least one liquid evacuation line 10 connected with an outlet port 4b of the second chamber 4 for receiving at least a dialysate filtered across the semipermeable membrane 5. A dialysis line 11 supplies a fresh dialysis liquid to an inlet 4a of the second chamber 4. A liquid check organ 12 may be used to selectively enable or inhibit a passage of liquid across the dialysis line 11, according to whether it is desired, or not, to have a purification by diffusive effect internally of the treatment unit 2.

In the example of FIG. 1, the dialysis line 11 and the infusion line 9 are part of a same supply line and the dialysis liquid and the replacement fluid are the same liquid crossing the supply line.

The apparatus 1 comprises sensor devices for determining the actual values assumed during treatment by parameters described herein below.

The apparatus 1 further comprises a control unit CPU connected with the sensor devices and configured to receive prescription values of said parameters to be reached in the patient P or to follow over a treatment time (T) and to obtain, through the sensor devices, the actual values of said parameters during the extracorporeal blood treatment. The apparatus 1 further comprises a user interface, not shown, provided with input and output devices, like a keyboard, a display, a touch screen, etc. connected to the control unit CPU.

### Parameters

The following parameters are considered (prescribed and/or detected and/or adjusted) by the apparatus 1 for extracorporeal blood treatment:
a first parameter (BV %) relating to a variation in blood volume of the patient P;
a second parameter (UFR; WLR; WL) relating to an ultrafiltration flow rate through the semipermeable membrane 5 or to a weight loss rate of the patient P or to an accumulated weight loss;
a third parameter (Cd, Na) relating to a conductivity of the liquid crossing the dialysis line 11 and the infusion line 9 or to a concentration of at least sodium and possible other substances (e.g. potassium, bicarbonate, calcium, magnesium, etc.) typically composing the liquid crossing the dialysis line 11 and the infusion line 9;
a fourth parameter (Q_{INF}, TMP) relating to a parameter selected from: an infusion flow rate of the replacement liquid crossing the infusion line 9 or a transmembrane pressure between the first chamber 3 and the second chamber 4.

### First Parameter—Variation of Blood Volume

The sensor devices of the apparatus 1 comprise a first sensor S1 for detecting the actual values of the first parameter, e.g. variation of blood volume (BV %) or an actual value of a parameter from which the variation of blood volume (BV %) may be calculated in relation to the blood of a patient P subjected to treatment. The blood volume variation sensor S1 may for example be optical and able to detect a variation in t optical properties of the blood crossing a calibrated portion of tube. For instance, a blood volume variation sensor S1 may calculate, through the control unit CPU, a percentage variation of the blood volume (BV %) circulating in the patient from start of hemodialysis treatment (or hemofiltration, or hemodiafiltration) based on the measurement of the concentration of hemoglobin in the blood, according to the known formula: BV %(t)=(HGB₀/HGBₜ)−1, where HGB₀ represents the concentration of hemoglobin at start of treatment and HGBₜ the concentration of hemoglobin at time t in which variation of the blood volume (BV %) is calculated. The hemoglobin concentration is calculated based on the variation of optic absorbance, at a predetermined wavelength, of the blood flowing in the blood removal line 6, across a tract of tube having the appropriate optical properties, previously characterised.

### Second Parameter—Ultrafiltration Flow Rate

The sensor devices of the apparatus 1 comprise at least a second sensor S2A, S2B for detecting the actual values of the ultrafiltration flow rate (UFR; second parameter) across the semipermeable membrane 5. For example, a flow sensor S2A may be active on the evacuation line 10 and a flow sensor S2B on the dialysis line 11 such as to provide the control unit CPU with the instant value of the respective flows and thus enable the control unit CPU to calculate an instant ultrafiltration flow. Alternatively, a differential sensor may be provided, active on the evacuation line 10 and dialysis line 11 and therefore able directly to provide a signal relating to the ultrafiltration flow rate UFR.

The sensor or sensors S2A, S2B may be volumetric sensors, mass sensors such as for example Coriolis sensors, weight sensors such as for example scales, pump revolution sensors, or sensors of yet another type: as the type of sensors usable is not significant and since the techniques and the sensors for detecting absolute or differential flow values are known and within the experience of the expert person in the field, no further details thereof are included in the present text.

### Second Parameter—Weight Loss Rate

The weight loss rate (WLR; second parameter) may be measured by subtracting the infusion rate (for example as described thereafter) from the ultrafiltration flow rate (for example as described above) as UFR=Q_{INF}+WLR.

As a further alternative, a sensor may be provided which is able directly to provide a signal which gives the weight loss rate (WLR): for example a sensor able to differentially measure the rate taken from the evacuation line 10 and to subtract the flow rate crossing the dialysis line 11 and/or the rate or rates of infusion 9. The sensor may be a mass flow sensor (for example a Coriolis sensor), volumetric, electromagnetic, ponderal (such as a scales able to weigh bags of fluid) or another type.

### Second Parameter—Weight Loss

The apparatus 1 may also determine the weight loss (WL; second parameter) over a time period, for example from start of the treatment up to an instant t: for example the control unit CPU may be programmed to integrate the weight loss rate (WLR) over the time. Alternatively, a weight loss sensor may be provided, for example a sensor configured to detect the variation in overall weight of a patient P during treatment or a sensor destined to directly detect the overall weight of the net fluid extracted from the patient P.

### Third Parameter—Conductivity or Sodium Concentration

The sensor devices of the apparatus 1 further comprise at least a third sensor S3 for detecting the actual values of the third parameter, i.e. conductivity or concentration of at least sodium (plus another substance, like potassium) that is to be monitored of the liquid crossing the dialysis line 11 and/or the infusion line 9. For instance, the conductivity or concentration sensor S3 may be located immediately downstream of a device for regulating a composition of dialysis liquid and/or replacement fluid, which will be more fully described in the following.

The sensor devices of the apparatus 1 further comprise a third sensor S38 for detecting the actual values of the third parameter, i.e. conductivity or concentration of at least sodium, in the dialysate downstream of the second chamber 4 which is located on the liquid evacuation line 10.

The apparatus 1 may also not comprise a conductivity or concentration sensor directly acting on the patient or on the extracorporeal blood circuit. In this case, the control procedure uses a mathematical model M representing a kinetics of solutes in a distribution volume V in the patient for iteratively calculating, at each control instant t, an equivalent sodium concentration value Na_{eq(t)}.

Note that by equivalent sodium concentration at instantt(Na_{eq(t)}) reference is made to the constant sodium concentration in the dialysis liquid that, if applied at the start of treatment up to an instant t, would lead to the same plasma sodium concentration in the patient as is obtained at the same instant t with the variation of sodium concentration or conductivity set by the control procedure up to time t. In this case, also the prescription values of sodium concentration are prescription values of equivalent sodium concentration.

### Fourth Parameter—Infusion Flow Rate

The sensor devices of the apparatus 1 further comprise at least a fourth sensor S4 for detecting the actual values of the infusion flow rate (Q_{INF}; fourth parameter) of the replacement liquid crossing the infusion line 9. The fourth sensor or sensors S4 may be volumetric sensor/s, mass sensor/s such as for example Coriolis sensor/s, weight sensor/s such as for example scales, pump revolution sensor/s or sensor/s of still other types: as the type of sensors usable is not significant and since the techniques and the sensors for detecting absolute or differential flow values are known and within the experience of the expert person in the field, no further details thereof are included in the present text. In the case illustrated in FIG. 1, the infusion flow rate sensor S4 is configured to determine a number of revolutions of an infusion pump 13, by sending a corresponding signal to the control unit CPU which is configured such as to calculate a flow rate along the respective infusion line 9.

### Regulating Device

The apparatus 1 comprises a regulating device for regulating the second parameter. The regulating device are connected to the control unit CPU and are active on at least one of the extracorporeal blood circuit 8 and the liquid evacuation line 10. In the example of FIG. 1, the regulating device comprises an ultrafiltration pump 14 located on the liquid evacuation line 10 and able to recall fluid from the second chamber 4. In other embodiments, the regulating device may comprise two pumps piloted differentially, such as two blood pumps located one upstream and one downstream of the treatment unit 2, or a plurality of pumps located on the lines and piloted such as to create an ultrafiltration flow across the membrane 5 or combinations of one or more pumps and valves appropriately arranged on the blood lines 6, 7 or liquid evacuation line 10. The control unit CPU is connected to the regulating device to pilot the pump or pumps such as to adjust the ultrafiltration flow rate (UFR) as will be detailed thereafter.

### Liquid Regulating Device

The apparatus 1 further comprises a liquid regulating device 30 for regulating the third parameter, i.e. the composition of the liquid crossing the supply line, i.e. of the dialysis liquid and/or of the replacement fluid.

In the example of FIG. 1, the liquid regulating device 30 comprises one, two or more containers of concentrate 15, 16, 17 located on respective injection lines 15a, 16a, 17a which are predisposed to supply substances such as electrolytes, buffer agents or others towards a preparation line 18 of the liquid located upstream of the supply line.

The concentrate containers 15, 16, 17 may comprise concentrates in the liquid state or solid state, for example powder. Injection pumps 15b, 16b, 17b may be present on the injection lines 15a, 16a, 17a to move the fluid along the respective injection line towards the preparation line 18 which collects the liquid, for example water, from a source 19. The source 19 may comprise a deionized water source or a source of ultra-pure liquid. The water collected from the source and possibly subjected to filtering stages 19a (not detailed as known and not relevant to the present invention) is provided with the required substances.

According to the embodiment shown in FIG. 1, the supply line comprises the infusion line 9 and the dialysis line 11 which depart both from the preparation line 18. Therefore, the dialysis fluid and the replacement fluid are the same liquid prepared by the preparation line 18 liquid and crossing the supply line. A further fluid check organ 12a may also be used to selectively enable or inhibit a passage of fluid across the infusion line 9.

The concentration or conductivity sensor S3, possibly added-to by further concentration or conductivity sensors S3A, S3C located on the preparation line 18, is able to provide the control unit CPU with a relative signal of conductivity or concentration of a predetermined substance (like sodium and/or potassium) of the fluid crossing the preparation line 18 such that the control unit CPU may act on the liquid regulating device and in particular on the pumps 15b, 16b, 17b in order to regulate the conductivity (Cd) or concentration, for example of sodium (Na) and/or potassium (K), of the liquid crossing the dialysis line 11 and/or the infusion line 9.

The infusion line 9 may also collect the fluid from a further source (for example a bag containing replacement fluid, not shown) independent with respect to the water source 19, while the preparation line 18 exclusively supplies the dialysis line 11.

### Procedures

The control unit CPU may comprise one or more digital units, for example microprocessors, or one or more analog units, or a special combination of digital and analog units. The control unit CPU is connected with the regulating devices, with the user interface, with the sensor devices and with the various actuator organs (blood pump 100, infusion pump 13, ultrafiltration pump 14, fluid check organs 12, 12A) located along the lines 6, 9, 10, 11 and is configured or programmed to perform the procedures described herein. In a case in which the control unit CPU is programmable, the control unit CPU is connected with a data support for storing instructions which, when performed by the control unit CPU, determine performing of the procedures which will be described herein below. The data support may comprise a mass data memory, for example optical or magnetic, a re-programmable memory (EPROM, FLASH) or a memory of another nature. In an aspect of the invention, the control unit CPU is programmed or configured such as to perform a “Procedure for estimating targets” and a “Control procedure” comprising the steps described herein below.

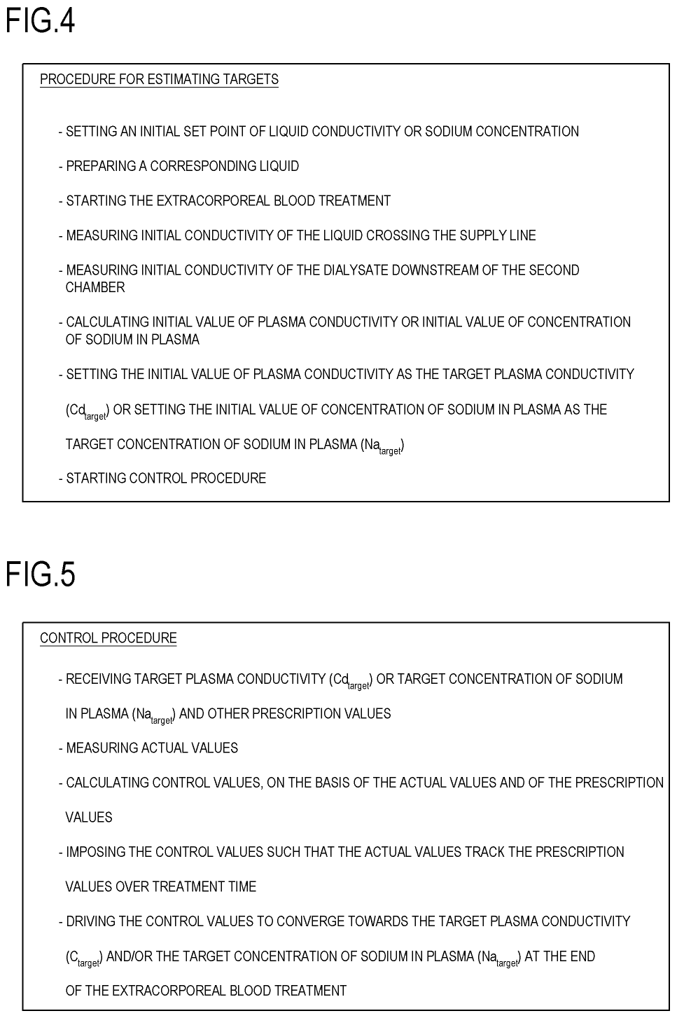

### Control Procedure (FIG. 5)

According to an example of the control procedure, in a first step, the control unit CPU receives, for instance via the user interface:
a treatment time (T),
an estimated or measured initial concentration of haemoglobin and/or an initial blood volume (BVᵢₙᵢₜ) of the patient;
a target variation in blood volume (BV %_{target}) to be reached at the end of the blood treatment;
a target ultrafiltration volume (UF_{target}) or target weight loss (WL_{target}) to be reached at the end of the blood treatment;
an initial set point of conductivity or sodium concentration (Cdᵢₙᵢₜ, Naᵢₙᵢₜ) of the liquid crossing the supply line 11;
a target plasma conductivity and/or a target concentration of sodium in plasma (Cd_{target}, Na_{target}) to be reached in the patient P at the end of the blood treatment.

The control unit CPU calculates a first parameter trajectory (BV %ₜᵣₐⱼ₍ₜ₎) from the estimated or measured initial concentration of haemoglobin and/or an initial blood volume of the patient P, the target variation in blood volume (BV %_{target}) and the treatment time (T). The control unit CPU also calculates a first parameter band delimited between an upper trajectory (above the first parameter trajectory (BV %ₜᵣₐⱼ₍ₜ₎) and a lower trajectory (below the first parameter trajectory (BV %ₜᵣₐⱼ₍ₜ₎), wherein a mid line of the first parameter band defines said first parameter trajectory (BV %ₜᵣₐⱼ₍ₜ₎).

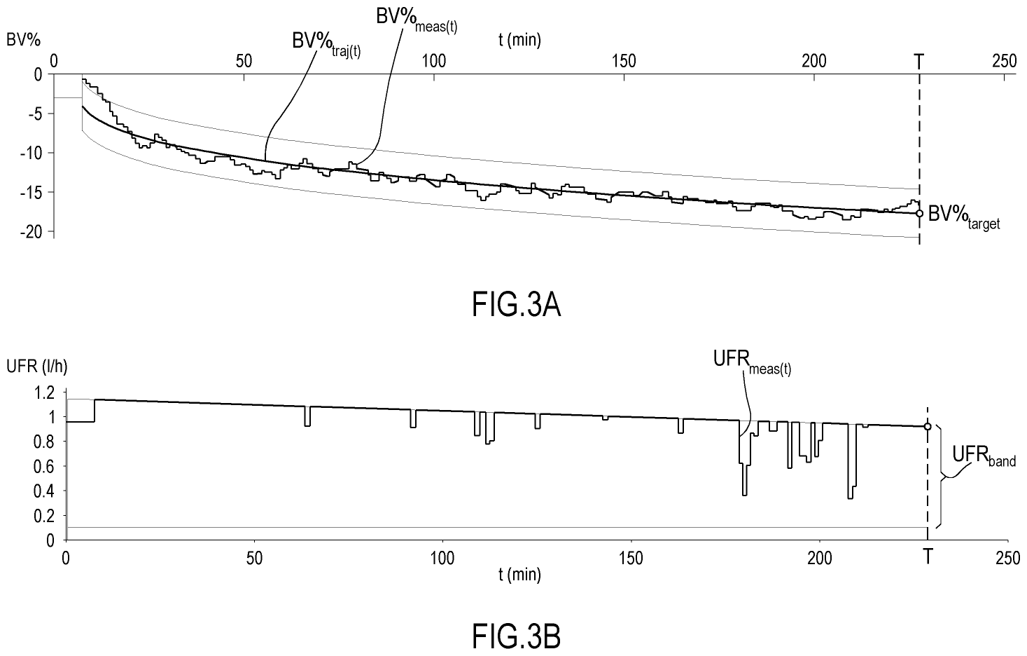

As shown in FIG. 3A, the first parameter trajectory (BV %ₜᵣₐⱼ₍ₜ₎) comprises a succession of prescription values of the variation in blood volume (BV %) to be followed over the treatment time. Usually, the first parameter trajectory (BV %ₜᵣₐⱼ₍ₜ₎) decreases over treatment time. The mid line of the fourth parameter band shown in FIG. 3A is in the middle, i.e. equally spaced from the upper trajectory and lower trajectory, but said mid line may also be eccentric compared to the upper trajectory and lower trajectory. The control unit CPU calculates, from the target ultrafiltration volume (UF_{target}) or target weight loss (WL_{target}) and the treatment time (T), a second parameter band or ultrafiltration flow rate band (UFR_{band}) delimited between an upper trajectory and a lower trajectory over the treatment time. Usually, the upper trajectory and a lower trajectory are straight lines slightly decreasing and converging one towards the other over treatment time, like in FIG. 38. The points of the the upper trajectory and the lower trajectory and comprised between the upper trajectory and the lower trajectory are prescription values of the UFR. The control unit CPU calculates, from the initial set point of conductivity or sodium concentration (Cdᵢₙᵢₜ, Naᵢₙᵢₜ), from the target plasma conductivity and/or the target concentration of sodium in plasma (Cd_{target}, Na_{target}) and the treatment time (T), a third parameter band or conductivity or sodium concentration band (Cd_{band}, Na_{band}) delimited between an upper trajectory and a lower trajectory over the treatment time. Usually, the upper trajectory and the lower trajectory are straight lines slightly decreasing and converging one towards the other over the treatment time, like in FIG. 3C. The points of the the upper trajectory and the lower trajectory and comprised between the upper trajectory and the lower trajectory are prescription values of the Cd or Na.

Blood treatment is started and the control unit CPU receives continuously and in real time during treatment (real time acquisition):
the actual values of the variation of blood volume (BV %ₘₑₐₛ₍ₜ₎) from the first sensor S1 (FIG. 3A);
the actual values of the ultrafiltration flow rate (UFRₘₑₐₛ₍ₜ₎) from the second sensors S2A, S2B (FIG. 38);
the actual values of the conductivity or sodium concentration (Cdₘₑₐₛ₍ₜ₎, Naₘₑₐₛ₍ₜ₎) in the liquid crossing the supply line 11 from the third sensor S3 (FIG. 3C).

As remarked before, the actual values of the sodium concentration may be values of equivalent sodium concentration value Na_{eq(t)} iteratively calculated as disclosed above.

The control unit CPU calculates, at temporally consecutive control instants and on the basis of the actual values and of the prescription values, the following control values to be set during a time interval after the instant in which the control is made:
a second parameter control value. i.e. ultrafiltration flow rate control value (UFR_{control(t)}); and/or
a third parameter control value, i.e. conductivity or sodium concentration control value (Cd_{control(t)}; Na_{control(t)}).

The control unit CPU commands the ultrafiltration pump 14 (regulating device) to impose the ultrafiltration flow rate control value (UFR_{control(t)}) during a time interval consecutive to the instant in which the control is made. In other words, the ultrafiltration pump 14 is adjusted such that the actual value of the ultrafiltration flow rate UFR_{meas(t+Δt)} at the time interval after the instant in which the control is made is equal or close to the ultrafiltration flow rate control value (UFR_{control(t)}). The control unit CPU commands the injection pumps 15b, 16b, 17b (liquid regulating device) to impose the conductivity or sodium concentration control value (Cd_{control(t)}; Na_{control(1)}) during a time interval consecutive to the instant in which the control is made. In other words, the injection pumps 15b, 16b, 17b are adjusted such that the actual values of the conductivity or sodium concentration (Cd_{meas(t+Δt)}, Na_{meas(t+Δt)}) at the time interval after the instant in which the control is made is equal or close to the conductivity or sodium concentration control value (Cd_{control(t)}; Na_{control(t)}).

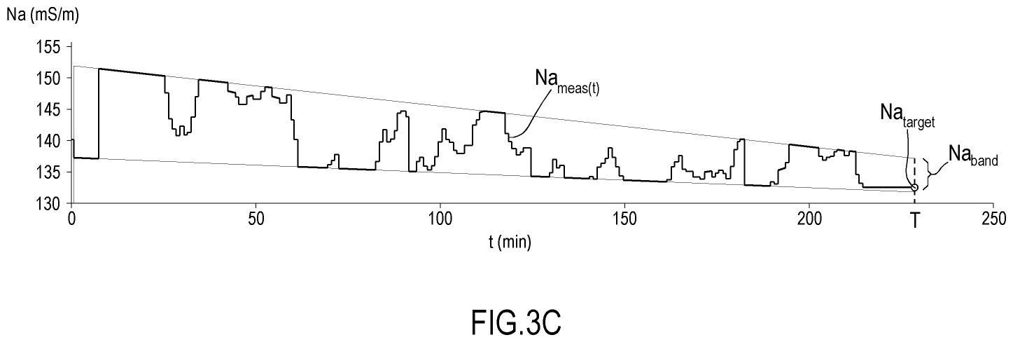

The ultrafiltration flow rate control value (UFR_{control(t)}) and the the conductivity or sodium concentration control value (Cd_{control(t)}; Na_{control(t)}) are calculated and imposed such that the actual values of the variation of blood volume (BV %ₘₑₐₛ₍ₜ₎) track the prescription values of the variation of blood volume (first parameter trajectory BV %ₜᵣₐⱼ₍ₜ₎) over the treatment time (T). For instance, the ultrafiltration flow rate control value (UFR_{control(t)}) and the the conductivity or sodium concentration control value (Cd_{control(t)}; Na_{control(t)}) are calculated and imposed such that the values of the variation of blood volume (BV %ₘₑₐₛ₍ₜ₎) follows the prescribed trajectory or is kept in a neighborough of said the prescribed trajectory or within the prescribed band (BV %ₜᵣₐⱼ₍ₜ₎, FIG. 4A). In addition, the ultrafiltration flow rate control value (UFR_{control(t)}; WLR_{control(t)}; WL_{control(t)}) is calculated to be within the ultrafiltration flow rate band (UFR_{band}) and the conductivity or sodium concentration control value is calculated to be within the conductivity or sodium concentration band (Cd_{band} Na_{band}), i.e. to keep the respective actual values within the respective bands, as shown in FIGS. 38 and 3C. The ultrafiltration flow rate control value (UFR_{control(t)}; WLR_{control(t)}; WL_{control(t)}) and/or the conductivity or sodium concentration control value (Cd_{control(t)}; Na_{control(t)}) may also be null, i.e. the control unit CPU may stop ultrafiltration and/or sodium administration.

The control values (UFR_{control(t)}; Cd_{control(t)}; Na_{control(t)}) may be calculated as follows.

The control unit CPU determines a first error parameter (ERR_BV_UF₍ₜ₎) on the basis of a difference between the actual value of the variation of blood volume (BV %ₘₑₐₛ₍ₜ₎) at the control instant (t) and a corresponding value on the first parameter trajectory (BV %ₜᵣₐⱼ₍ₜ₎) and a difference between an actual value of the second parameter (UFRₘₑₐₛ₍ₜ₎; WLRₘₑₐₛ₍ₜ₎; WLₘₑₐₛ₍ₜ₎) at the control instant (t) and a corresponding value of the second parameter band. The control unit CPU determines a second error parameter (ERR_BV_Na₍ₜ₎) on the basis of a difference between the actual value of the variation of blood volume (BV %ₘₑₐₛ₍ₜ₎) at the control instant (t) and a corresponding value on the first parameter trajectory (BV %ₜᵣₐⱼ₍ₜ₎) and a difference between an actual value of the third parameter (Cdₘₑₐₛ₍ₜ₎; Naₘₑₐₛ₍ₜ₎) at the control instant (t) and a corresponding value of the third parameter band. Then, the control unit CPU calculates a first value of the second parameter control value (UFR_{control(t)}; WLR_{control(t)}; WL_{control(t)}) on the basis of the first error parameter (ERR_BV %_UF₍ₜ₎) and of the actual value of the second parameter (UFR_{meas(t-Δt)}); WLR_{meas(t-Δt)}); WL_{meas(t-Δt)}) relating to a preceding control instant and calculates a first value of the third parameter control value (Cd_{control(t)}; Na_{control(t)} on the basis of the second error parameter (ERR_BV %_Na₍ₜ₎) and of the actual value of the third parameter(Cd_{meas(t-Δt)}, Na_{meas(t-Δt)} relating to a preceding control instant. Near the end of the extracorporeal blood treatment, the control procedure comprises: driving the third parameter control values to converge towards the target plasma conductivity and/or the target concentration of at least sodium in plasma such that at the end of the extracorporeal blood treatment the third parameter control value is close or substantially equal to the target plasma conductivity (Cd_{target}) and/or the target concentration of sodium in plasma (Na_{target}).

The control procedure above described is automatically managed by the control unit CPU. In case, despite the automatic control procedure, the actual values of the variation of blood volume (BV %ₘₑₐₛ₍ₜ₎) move away from the respective prescription values (move away from a prescribed trajectory or move outside a prescribed band or move above or below a prescribed threshold), the control unit CPU is programmed to issue an alarm signal to alert the staff and/or to stop the blood treatment.

### Procedure for Estimatina Targets (FIG. 4)

In an aspect of the invention, the control unit CPU is programmed or configured such as to perform, at least at the beginning of the extracorporeal blood treatment, an estimation procedure for automatically estimating/calculating said target plasma conductivity and/or said target concentration of sodium in plasma (Cd_{target}, Na_{target}) to be reached in the patient at the end of the blood treatment. The control procedure described above starts when the procedure for estimating is completed.

The procedure for estimating comprises the steps described herein below.

First, the control unit CPU sets the initial set point of conductivity or sodium concentration (Cdᵢₙᵢₜ, Naᵢₙᵢₜ) of the liquid crossing the supply line 11. The set point is calculated before starting the blood circulation (i.e. before starting the extracorporeal blood treatment). In detail, the control unit CPU is configured to set the parameter value for the liquid at the initial set point so that a liquid conductivity matches a first estimate of the plasma conductivity of the blood and may be calculated as a function of the type of extracorporeal blood treatment mode, i.e. hemodialysis (HD), hemofiltration (HF) or hemodiafiltration (HDF).

The initial set point of sodium concentration (Nam) of the liquid crossing the supply line 11 may be calculated as a function of: a concentration of one or more further substances (in addition to sodium) in the liquid crossing the supply line, an estimated plasma concentration of one or more substances, molar conductivities of one or more substances in the liquid crossing the supply line, at least one flow rate, as the dialysate flow rate at the outlet of the second chamber, an efficiency parameter of the treatment unit, like an urea clearance of the treatment unit. For instance, in hemodialysis (HD) mode, the control unit CPU is configured to calculate the initial set point of sodium concentration to be set in the liquid crossing the supply line before the start of the treatment using the following relationship:

$${c}_{di , Na , start} = α * {c}_{pw , Na} + \frac{1}{{M}_{{κ}_{NaCl}}} ( {M}_{{κ}_{{NaHCO}_{3}}} - {{M}_{κ}}_{NaCl} ) ( \frac{1}{α} * {c}_{pw , {HCO}_{3}} - {c}_{di , {HCO}_{3}} ) + \frac{1}{{{M}_{κ}}_{NaCl}} ( {{M}_{κ}}_{NaAc} - {{M}_{κ}}_{NaCl} ) ( \frac{1}{α} * {c}_{pw , Ac} - {c}_{di , Ac} ) + \frac{{{M}_{κ}}_{KCl}}{{{M}_{κ}}_{NaCl}} ( α * {c}_{pw , K} - {c}_{di , K} ) + \frac{1}{{{M}_{κ}}_{NaCl}} \frac{{Q}_{do}}{{K}_{u}} {κ}_{rest 3} \qquad ( Equation 1 )$$

wherein the used symbols meaning is clarified in the following table 1.

TABLE 1

| Name | Description | Unit |
|---|---|---|
| Q_{do} | Dialysate flow rate at the outlet of the | mL/min |
| | second chamber | |
| Kᵤ | Treatment unit clearance for urea | mL/min |
| c_{d} [symbol] _{K} | Concentration of potassium ions in the | mmol/L |
| | liquid crossing the supply line as | |
| | determined by the used concentrate | |
| c_{d} [symbol] _{Ac} | Concentration of acetate in the liquid | mmol/L |
| | crossing the supply line as determined | |
| | by the used concentrate | |
| c_{ow} [symbol] _{Na} | Estimated or measured pre-dialysis | mmol/L |
| | concentration of sodium ions in plasma | |
| | water | |
| c_{ow} [symbol] _{HCO} [symbol] | Estimated or measured pre-dialysis | mmol/L |
| | concentration of bicarbonate anions | |
| | in plasma water | |
| c_{ow} [symbol] _{Ac} | Estimated or measured pre-dialysis | mmol/L |
| | concentration of acetate anions in | |
| | plasma water | |
| c_{ow} [symbol] _{K} | Estimated or measured pre-dialysis | mmol/L |
| | concentration of potassium ions in | |
| | plasma water | |
| kᵣₑₛₜ₃ | Conductivity contribution from lesser | mS/cm |
| | solutes 3 | |
| M [symbol] _{NaHCO} [symbol] | Molar conductivity of sodium | L · mS/ |
| | bicarbonate at ionic strength 150 mM | mol · cm |
| M [symbol] _{NaCl} | Molar conductivity of sodium chloride | L · mS/ |
| | at ionic strength 150 mM | mol · cm |
| M [symbol] _{NaAc} | Molar conductivity of sodium acetate | L · mS/ |
| | at ionic strength 150 mM | mol · cm |
| M [symbol] _{KCl} | Molar conductivity of potassium | L · mS/ |
| | chloride at ionic strength 150 mM | mol · cm |
| [symbol] | Donnan factor | |

[symbol]

indicates data missing or illegible when filed

In case of HDF post-dilution mode is selected, the same equation (1) applies. Differently from HD mode, treatment unit clearance Kᵤ is calculated for the HDF post-dilution mode. In case of HDF pre-dilution mode is selected, the dilution of the blood before it enters the treatment unit should be taken into account. The equations for HF post-dilution mode are the same as for to HD, but the treatment unit clearance is substituted by the dialysate flow rate at treatment unit outlet, i.e. Kᵤ=Q_{db}. In case of HF pre-dilution mode is selected, the dilution of the blood before it enters the dialyzer should be taken into account.

Once the sodium initial set point has been calculated, a corresponding liquid is prepared by the control unit CPU driving the preparation line 18. Then the extracorporeal blood treatment is started by circulating the liquid through the supply line and by circulating blood through the extracorporeal blood circuit 8.

Depending on the selected treatment mode, the dialysis fluid is directed:
to the second chamber 4 of the treatment unit 2 only so as to exchange with blood (HD mode);
to the second chamber 4 of the treatment unit 2 so as to exchange with blood and infused into the blood circuit 8 (HDF mode);
to the blood circuit 8 only (HF mode).

Correspondingly, blood is withdrawn from the patient P and circulated in the extracorporeal blood circuit 8 and particularly is circulated through the first chamber 3 of the filtration unit 2.

Then at least one, and in general a plurality, of consecutive initial values of the third parameter (in the example, the conductivity) of the dialysate downstream of the second chamber 4 are measured at the beginning of the treatment through the third sensor S38.

The dialysate conductivity will change initially, due to e.g. dynamics when treatment is started (e.g. leaving bypass conditions) or when blood flow is ramped up. However, it is expected to stabilize within few (e.g. 4) minutes. The measurement is made as soon as stability criteria are fulfilled. The control unit CPU is configured to validate and further process the measurement of an initial value of the conductivity of the dialysate as soon as the diffusion process in the treatment unit 2 reaches stable conditions.

In order to minimize the time needed to reach stability conditions, changes in the flow rate of liquid crossing the supply line and in bicarbonate prescription may be prevented during this preliminary sodium identification phase. Changes in blood flow, ultrafiltration flow rate or bypass are vice versa generally allowed, but they will delay stability. Moreover, it is not possible to change the concentrate combination type after the treatment is started.

An initial conductivity of the liquid crossing the supply line upstream the second chamber 4 is either measured or taken as the set value for liquid conductivity. In general, the initial conductivity of the liquid may be measured through the third sensor S3.

The initial setting of the sodium concentration calculated or determined as above stated to be as close as possible to the expected plasma conductivity (equation 1) may be optional, meaning that the method for estimating the initial plasma conductivity may be performed even if the sodium content of the sodium concentration to be set in the liquid crossing the supply line is initially simply set by the operator.

The control unit CPU calculates an initial value of plasma conductivity based on the measured initial parameter value of the dialysate (i.e. based on conductivity or concentration measurement of dialysate on the treatment unit outlet) and on the corresponding parameter value of the liquid crossing the supply line (e.g. conductivity or concentration). During the start of the treatment and particularity during circulating the liquid through the second chamber 4 and/or in the infusion line 9 up to measuring the initial value of the parameter of the dialysate downstream of the second chamber 4 used for the calculating of the initial plasma conductivity, the liquid conductivity (or concentration of sodium) is kept substantially constant. In other words, the calculation of the initial plasma conductivity is performed with no conductivity step.

After calculating the initial value of plasma conductivity, the control unit CPU is configured to drive a liquid regulating device 30 to change the composition of the liquid crossing the supply line to reach a liquid conductivity substantially equal to the initial value of plasma conductivity (isoconductivity).

According to a first embodiment, the control unit CPU is programmed to calculate the initial value of plasma conductivity based on the sum of at least the initial conductivity of the dialysate plus a difference between inlet and outlet conductivity at the treatment unit 2, or dialyzer, weighted by a factor of the dialysate flow rate. In more detail, the difference between inlet and outlet conductivity at the treatment unit 2 is weighted by a factor of the blood flow rate in the blood lines too.

According to the first embodiment, the control unit CPU is configured to calculate the initial value of plasma conductivity using the following formula:

$${κ}_{p , 1}^{′} = {κ}_{0 , d o} + \frac{{Q}_{d o}}{{Q}_{Bset}} ( {κ}_{0 , do} - {κ}_{0 , di} ) \qquad ( Equation 2 )$$

The significance of the denotations above is given in the following table 2.

TABLE 2

| | |
|---|---|
| κ_{p, 1} | plasma conductivity first estimate; |
| Q_{do} | dialysate flow rate at the treatment unit outlet; |
| Q_{bset} | set blood flow rate at the treatment unit inlet; |
| [symbol] _{0, di} | liquid conductivity at the treatment unit inlet |
| | for a pure electrolyte solution; |
| [symbol] _{0, do} | dialysate conductivity at the treatment unit |
| | outlet for a pure electrolyte solution. |

It is worth to underline that during the above described calculation of the initial plasma conductivity (Equation 2), the liquid circulates through the second chamber 4 and/or is infused into the blood circuit 8 (depending on selected HD/HF/HDF mode) maintaining the liquid parameter value substantially constant.

In a second embodiment, the control unit CPU is programmed to calculate the initial plasma conductivity based on the sum of at least the initial conductivity of the fresh liquid plus a difference between inlet and outlet conductivity at the treatment unit 2 weighted by a factor of the dialysate flow rate. In more detail the difference between inlet and outlet conductivity at the treatment unit 2, or dialyzer, is weighted by a factor of the dialyzer clearance too.

According to the second embodiment the control unit CPU is configured to calculate the plasma conductivity using the following formula:

$${\begin{matrix}{κ}_{p , 1}^{″} = {κ}_{0 , d i} + \frac{{Q}_{d o}}{{K}_{u}} ( {κ}_{0 , do} - {κ}_{0 , di} ) & ( Equation 3 )\end{matrix}}_{}$$

The significance of the denotations above is given in the following table 3.

TABLE 3

| | |
|---|---|
| κ_{p, 1} | plasma conductivity first estimate; |
| Q_{do} | dialysate fluid flow rate at the treatment unit outlet; |
| Kᵤ | treatment unit clearance for urea; |
| [symbol] _{0, di} | liquid conductivity at the treatment unit inlet for |
| | a pure electrolyte solution; |
| [symbol] _{0, do} | dialysate conductivity at the treatment unit outlet for |
| | a pure electrolyte solution. |

Of course, both formulas (2) and (3) for estimation of plasma conductivity may be iteratively applied, meaning that the newly calculated estimate of plasma conductivity (k_{p,1}) is imposed to the liquid (through the liquid regulating device 30) and a new estimate (k_{p,2}) again calculated after taking measures of the conductivity at the inlet and outlet of the treatment unit 2 as soon as stable conditions are reached.

Once the initial value of plasma conductivity has been estimated through the “Procedure for estimating targets”, said initial value of plasma conductivity is set or imposed as the target plasma conductivity (Cd_{target}) to be reached in the patient P at the end of the extracorporeal blood treatment and used in the “Control procedure” formerly disclosed.

According to a different embodiment, once the initial value of plasma conductivity has been estimated through the “Procedure for estimating targets”, the initial value of concentration of sodium in plasma is derived from the initial value of plasma conductivity and said initial value of concentration of sodium in plasma is set or imposed as the target concentration of sodium in plasma (Na_{target}) to be reached in the patient P at the end of the extracorporeal blood treatment and used in the “Control procedure” formerly disclosed.

Said initial value of concentration of sodium in plasma (or target concentration of sodium in plasma Na_{target}) may be set as the concentration of sodium in the liquid crossing the supply line when the liquid conductivity matches the initial value of plasma conductivity (or target plasma conductivity Cd_{target}), i.e. when the liquid tonicity is not changed during its passage through the treatment unit 2 and said liquid tonicity matches the tonicity of plasma.

According to other embodiments, adjustment factors are applied to the estimated initial value of plasma conductivity or estimated initial value of concentration of sodium in plasma to obtain corrected values of plasma conductivity or concentration of sodium in plasma and then the target plasma conductivity (Cd_{target}) or the target concentration of sodium in plasma (Na_{target}).

These adjustment factors may be chosen by a doctor. In other words, the estimation of the initial patients plasma sodium concentration/conductivity may be used as reference for the doctor to eventually decide to correct the target final patients plasma sodium concentration/conductivity.

These adjustment factors may also be calculated. In this instance, the target final patient's plasma sodium concentration/conductivity is the sum of a main contribution term based on/function of the plasma conductivity and as a function of the adjustment factor. According to an ambodiment, the target concentration of sodium in plasma is set as the sum of the concentration of sodium in the liquid crossing the supply line when the liquid conductivity matches the initial value of plasma conductivity (main contribution term) and of a concentration adjustment factor, according to the following general formula:

$${c}_{di , Na , set} = {c}_{di , Na , {κ}_{p , pre}} + {c}_{di , Na , adj} \qquad ( Equation 4 )$$

wherein
C_{di,Na, κ}_{_{p,pre}} is the main contribution term of the liquid crossing the dialysis line at isocondictive state; and
C_{di,Na,adj} is the sodium concentration set point adjustment relative to an isoconductive state, which would provide a treatment which may be, for example, chosen in the group including isotonic dialysis, isonatric dialysis, and isonatrikalemic dialysis.

The main contribution term usually affects the initial value of concentration of sodium in plasma for 80%-90% of the initial value of concentration of sodium in plasma while the concentration adjustment factor contributes to the initial value of concentration of sodium in plasma for 10%-15%, of the initial value of concentration of sodium in plasma.

Even if the initial plasma sodium concentration/conductivity and the final/target plasma sodium concentration/conductivity (Cd_{target}, Na_{target}) are known/set through the “Procedure for estimating”, during the extracorporeal blood treatment, the values of the actual sodium concentration/conductivy of the liquid and of plasma continuously change according to the “Control procedure”. Only at the end of the extracorporeal blood treatment, the control unit CPU drives the liquid regulating device 30 to change the composition of the liquid crossing the supply line to reach the target concentration of sodium in plasma Na_{target} or the target plasma conductivity Cd_{target}.

During the extracorporeal blood treatment, the control unit may also be configured to check the conductivity of plasma in patient and/or the concentration of plasma sodium in patient to monitor treatment evolution. Values of plasma conductivity and/or of concentration of sodium in plasma may be calculated through a procedure similar to the procedure for estimating targets disclosed above.

For instance, the control unit CPU calculates the plasma conductivity from measured values of conductivity in the liquid evacuation line and in the supply line and through Equations 2 or 3 above outlined.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the scope of the appended claims.

1-22. (canceled)

23. An apparatus for extracorporeal blood treatment, the apparatus comprising:
a treatment unit having a first chamber and a second chamber separated from one another by a semipermeable membrane;
a blood removal line connected to an inlet port of the first chamber and predisposed to remove blood from a patient;
a blood return line connected to an outlet port of the first chamber and predisposed to return treated blood to the patient, the blood removal line, the blood return line, and the first chamber being part of an extracorporeal blood circuit;
a liquid evacuation line connected to an outlet port of the second chamber;
a supply line connected to an inlet port of the second chamber and/or connected to the extracorporeal blood circuit;
sensor devices for detecting actual values of parameters of the extracorporeal blood treatment; and
a control unit connected to the sensor devices and configured to receive prescription values of the parameters to be reached in the patient or to follow over a treatment time and to obtain, through the sensor devices, the actual values of the parameters during the extracorporeal blood treatment, wherein the parameters comprise:
a first parameter relating to a variation in blood volume of the patient,
a second parameter relating to an ultrafiltration flow rate through the semipermeable membrane, a weight loss rate of the patient, or an accumulated weight loss, and
a third parameter chosen from a group consisting of: a conductivity of a liquid crossing the supply line, a conductivity-related parameter of the liquid crossing the supply line, a concentration of sodium in the liquid crossing the supply line, and a concentration-related parameter of sodium in the liquid crossing the supply line,
wherein the control unit is configured to perform, at temporally consecutive control instants, a control procedure comprising:
calculating, on the basis of the actual values and of the prescription values, the following control values to be set during a time interval after the instant in which the control is made:
a second parameter control value, and
a third parameter control value, and
imposing at least one of the second parameter control value and third parameter control value during the time interval consecutive to the instant in which the control is made such that the actual values of the first parameter track the prescription values of the first parameter over the treatment time,
wherein the prescription value of the third parameter comprises a target plasma conductivity and/or a target concentration of sodium in plasma to be reached in the patient in the treatment time, and
wherein the control unit is further configured to perform, at least at the beginning of the extracorporeal blood treatment, a procedure for estimating the target plasma conductivity and/or the target concentration of sodium in plasma.

24. The apparatus of claim 23, wherein the control procedure starts when the procedure for estimating is completed.

25. The apparatus of claim 23, further comprising a preparation line of the liquid crossing the supply line located upstream of and connected to the supply line, wherein the procedure for estimating comprises:
setting an initial set point of the third parameter of the liquid crossing the supply line and preparing a corresponding liquid through the preparation line;
after setting the initial set point, starting extracorporeal blood treatment by circulating the liquid through the supply line and by circulating blood through the extracorporeal blood circuit;
measuring an initial value of the third parameter of the dialysate in the liquid evacuation line at the beginning of the extracorporeal blood treatment;
calculating, based on the measured initial value of the third parameter of the dialysate in the liquid evacuation line, an initial value of plasma conductivity and/or an initial value of concentration of sodium in plasma; and
setting the initial value of plasma conductivity as the target plasma conductivity and/or setting the initial value of concentration of sodium in plasma as the target concentration of sodium in plasma.

26. The apparatus of claim 25, wherein the procedure for estimating further comprises:
measuring an initial value of the third parameter of the liquid crossing the supply line at the beginning of the extracorporeal blood treatment; and
calculating the initial value of plasma conductivity and/or the initial value of concentration of sodium in plasma based also on the measured initial value of the third parameter of the liquid crossing the supply line or on the initial set point of the third parameter of the liquid crossing the supply line.

27. The apparatus of claim 25, wherein the procedure for estimating further comprises:
applying a conductivity adjustment factor to the initial value of plasma conductivity to obtain a corrected value of plasma conductivity and/or applying a concentration adjustment factor to the initial value of concentration of sodium in plasma to obtain a corrected value of concentration of sodium in plasma; and
setting the corrected value of plasma conductivity as the target plasma conductivity and/or setting the corrected value of concentration of sodium in plasma as the target concentration of sodium in plasma.

28. The apparatus according to claim 25, wherein the initial set point of the third parameter is set by an operator or the control unit is configured to calculate the initial set point of the third parameter to match a first estimate of the plasma conductivity of the patient.

29. The apparatus of claim 28, wherein the control unit is configured to calculate the initial set point of the third parameter as a function of a type of extracorporeal blood treatment mode.

30. The apparatus of claim 25, wherein the control unit is configured to measure the initial value of the third parameter of the dialysate as soon as the exchange process in the treatment unit reaches stable conditions, the control unit being configured to determine reaching of stable conditions for the exchange process.

31. The apparatus of claim 25, wherein the control unit is configured to measure a plurality of consecutive initial values of the third parameter of the dialysate until the values are stabilized to provide the initial value of the third parameter.

32. The apparatus of claim 25, wherein the procedure for estimating comprises:
after calculating the initial value of plasma conductivity, the control unit is configured to drive a liquid regulating device for regulating a composition of the liquid crossing the supply line to change the composition of the liquid crossing the supply line to reach a liquid conductivity substantially equal to the initial value of plasma conductivity.

33. The apparatus of claim 32, wherein the procedure for estimating comprises: after setting the liquid conductivity substantially equal to the initial value of plasma conductivity, the control unit is configured to execute a second calculating step, based on a second determined initial conductivity of the dialysate and on a second corresponding conductivity of the liquid in the supply line, of a second estimate of the initial value of plasma conductivity, the calculating the second estimate being performed maintaining the liquid conductivity substantially constant and substantially equal to the calculated plasma conductivity, wherein, after calculating the second estimate of the initial value of plasma conductivity, the control unit is configured to drive the liquid regulating device to change the composition of the liquid and to set the liquid conductivity substantially equal to said second estimate.

34. The apparatus of claim 25, wherein the initial value of concentration of sodium in plasma is derived from the initial value of plasma conductivity.

35. The apparatus of claim 34, wherein the target concentration of sodium in plasma is set as a concentration of sodium in the liquid crossing the supply line when the liquid conductivity matches the initial value of plasma conductivity.

36. The apparatus of claim 25, wherein the initial set point of the third parameter is the initial set point of sodium concentration in the liquid crossing the supply line, wherein the control unit is configured to calculate the initial set point of sodium concentration in the liquid crossing the supply line as a function of the concentration of a further substance in the liquid crossing the supply line or as a function of an estimated plasma concentration of a substance or as a function of the weighted difference in concentration of a further substance in the liquid crossing the supply line and in the plasma or as a function of the molar conductivities of a substance in the liquid crossing the supply line.

37. The apparatus of claim 25, wherein the control unit is configured to calculate the initial set point of the third parameter in the liquid crossing the supply line as a function of the dialysate flow rate at the outlet of the second chamber and/or as a function of a clearance of the treatment unit.

38. The apparatus of claim 25, wherein calculation of the initial value of plasma conductivity and/or the initial value of concentration of sodium in plasma is done with an iterative method.

39. The apparatus of claim 23, wherein at least one time during the extracorporeal blood treatment, the control unit is configured to check plasma conductivity and/or concentration of sodium in plasma.

40. The apparatus of claim 23, wherein the control procedure comprises the following sub-steps:
receiving the target plasma conductivity and/or the target concentration of sodium in plasma;
receiving the treatment time; and
determining, on the basis of the target plasma conductivity and/or on the base of the target concentration of sodium in plasma, a third parameter band delimited between an upper trajectory and a lower trajectory over the treatment time,
wherein the calculated third parameter control values are kept within the third parameter band.

41. The apparatus according to claim 40, wherein the prescription values of the first parameter define a first parameter trajectory and tracking the prescription values of the first parameter comprises: keeping or moving the actual values of the first parameter on the first parameter trajectory or in a neighborhood of the first parameter trajectory, wherein the prescription values of the second parameter define a second parameter band delimited between an upper trajectory and a lower trajectory over the treatment time,
wherein calculating the second parameter control value comprises:
determining a first error parameter on the basis of:
a difference between an actual value of the first parameter at the control instant and a corresponding value on the first parameter trajectory, and
a difference between an actual value of the second parameter at the control instant and a corresponding value of the second parameter band;
calculating the second parameter control value on the basis of the first error parameter and of the actual value of the second parameter relating to a preceding control instant,
wherein calculating the third parameter control value comprises:
determining a second error parameter on the basis of:
a difference between an actual value of the first parameter at the control instant and a corresponding value on the first parameter trajectory, and
a difference between an actual value of the third parameter at the control instant and a corresponding value of the third parameter band; and
calculating the third parameter control value on the basis of the second error parameter and of the actual value of the third parameter relating to a preceding control instant.

42. The apparatus of claim 23, wherein, near an end of the extracorporeal blood treatment, the control procedure comprises: driving the third parameter control values to converge towards the target plasma conductivity and/or the target concentration of sodium in plasma.

43. The apparatus according to claim 42, wherein, at the end of the extracorporeal blood treatment, the control procedure comprises: setting the third parameter control value close or substantially equal to the target plasma conductivity and/or the target concentration of sodium in plasma.

44. The apparatus of claim 23, wherein the actual values of the third parameter are values of concentration of sodium in the liquid crossing the supply line;
wherein the control procedure uses a mathematical model, representing kinetics of the solutes in a distribution volume in the patient, in order to determine equivalent sodium concentration values, wherein by equivalent sodium concentration at instant t is intended the constant sodium concentration in the liquid crossing the supply line which, if it were applied at a start of treatment up to an instant, would lead to the same plasma sodium concentration in the patient as is obtained at the same instant with the variation in sodium concentration or conductivity imposed by the control procedure up to time,
the control procedure using the equivalent sodium concentration values as actual values of the third parameter for the determination of the control values.

45. An apparatus for extracorporeal blood treatment, comprising:
a treatment unit having a first chamber and a second chamber separated from one another by a semipermeable membrane;
a blood removal line connected to an inlet port of the first chamber and predisposed to remove blood from a patient;
a blood return line connected to an outlet port of the first chamber and predisposed to return treated blood to the patient, the blood removal line, the blood return line, and the first chamber being part of an extracorporeal blood circuit;
a liquid evacuation line connected to an outlet port of the second chamber;
a supply line connected to an inlet port of the second chamber and/or connected to the extracorporeal blood circuit;
a preparation line of the liquid crossing the supply line located upstream of and connected to the supply line;
sensor devices for detecting actual values of parameters of the extracorporeal blood treatment; and
a control unit connected to the sensor devices and configured to receive prescription values of the parameters to be reached in the patient or to follow over a treatment time and to obtain, through the sensor devices, the actual values of the parameters during the extracorporeal blood treatment, wherein the parameters comprise:
a first parameter relating to a variation in blood volume of the patient,
a second parameter relating to an ultrafiltration flow rate through the semipermeable membrane or to a weight loss rate of the patient or to an accumulated weight loss, and
a third parameter chosen from a group consisting of: a conductivity of a liquid crossing the supply line, a conductivity-related parameter of the liquid crossing the supply line, a concentration of sodium in the liquid crossing the supply line, and a concentration-related parameter of sodium in the liquid crossing the supply line,
wherein the control unit is configured to perform, at temporally consecutive control instants, a control procedure comprising:
calculating, on the basis of the actual values and of the prescription values, the following control values to be set during a time interval after the instant in which the control is made:
a second parameter control value, and
a third parameter control value, and
imposing at least one of the second parameter control value and third parameter control value during the time interval consecutive to the instant in which the control is made such that the actual values of the first parameter track the prescription values of the first parameter over the treatment time,
wherein the prescription value of the third parameter comprises a target plasma conductivity and/or a target concentration of sodium in plasma to be reached in the patient in the treatment time, and
wherein the control unit is further configured to perform, at least at a beginning of the extracorporeal blood treatment, a procedure for estimating the target plasma conductivity and/or the target concentration of sodium in plasma comprising:
setting an initial set point of the third parameter of the liquid crossing the supply line and preparing a corresponding liquid through the preparation line,
after setting the initial set point, starting extracorporeal blood treatment by circulating the liquid through the supply line and by circulating blood through the extracorporeal blood circuit,
measuring an initial value of the third parameter of the dialysate in the liquid evacuation line at the beginning of the extracorporeal blood treatment,
calculating, based on the measured initial value of the third parameter of the dialysate in the liquid evacuation line, an initial value of plasma conductivity and/or an initial value of concentration of sodium in plasma, wherein the control unit is configured to calculate the initial set point of the third parameter in the liquid crossing the supply line as a function of a dialysate flow rate at the outlet of the second chamber and/or as a function of a clearance of the treatment unit,
setting the initial value of plasma conductivity as the target plasma conductivity and/or setting the initial value of concentration of sodium in plasma as the target concentration of sodium in plasma, and
after calculating the initial value of plasma conductivity, driving a liquid regulating device for regulating a composition of the liquid crossing the supply line to change said composition of the liquid crossing the supply line to reach a liquid conductivity substantially equal to the initial value of plasma conductivity.
